SEQUENCE LISTING (33 sequences)

SEQ ID NO: 1 - protein, 7 aa, Artificial sequence
  synthetic
SNYLYDN

SEQ ID NO: 2 - protein, 9 aa, Artificial sequence
  synthetic
REFPFYGDY

SEQ ID NO: 3 - protein, 6 aa, Artificial sequence
  synthetic
EKHHSH

SEQ ID NO: 4 - protein, 38 aa, Artificial sequence
  synthetic
DSHEKRHHGYRRKFHEKHHSHREFPFYGDYGSNYLYDN

SEQ ID NO: 5 - protein, 27 aa, Artificial sequence
  synthetic
RKFHEKHHSHREFPFYGDYGSNYLYDN

SEQ ID NO: 6 - protein, 32 aa, Artificial sequence
  synthetic
DSHAKRHHGYKRKFHEKHHSHRGYRSNYLYDN

SEQ ID NO: 7 - protein, 15 aa, Artificial sequence
  synthetic
EKHHSHREFPFYGDY

SEQ ID NO: 8 - protein, 17 aa, Artificial sequence
  synthetic
REFPFYGDYGSNYLYDN

SEQ ID NO: 9 - protein, 19 aa, Artificial sequence
  synthetic
SHREFPFYGDYGSNYLYDN

SEQ ID NO: 10 - protein, 21 aa, Artificial sequence
  synthetic
HHSHREFPFYGDYGSNYLYDN

SEQ ID NO: 11 - protein, 23 aa, Artificial sequence
  synthetic
EKHHSHREFPFYGDYGSNYLYDN

SEQ ID NO: 12 - protein, 25 aa, Artificial sequence
  synthetic
FHEKHHSHREFPFYGDYGSNYLYDN

SEQ ID NO: 13 - protein, 19 aa, Artificial sequence
  synthetic
RKFHEKHHSHREFPFYGDY

SEQ ID NO: 14 - protein, 15 aa, Artificial sequence
  synthetic
EKHHSHREFPFYGDY

SEQ ID NO: 15 - protein, 21 aa, Artificial sequence
  synthetic
RKFHEKHHSHREFPFYGDYGS

SEQ ID NO: 16 - protein, 17 aa, Artificial sequence
  synthetic
EKHHSHREFPFYGDYGS

SEQ ID NO: 17 - protein, 23 aa, Artificial sequence
  synthetic
RKFHEKHHSHREFPFYGDYGSNY

SEQ ID NO: 18 - protein, 19 aa, Artificial sequence
  synthetic
EKHHSHREFPFYGDYGSNY

SEQ ID NO: 19 - protein, 25 aa, Artificial sequence
  synthetic
RKFHEKHHSHREFPFYGDYGSNYLY

SEQ ID NO: 20 - protein, 21 aa, Artificial sequence
  synthetic
EKHHSHREFPFYGDYGSNYLY

SEQ ID NO: 21 - protein, 17 aa, Artificial sequence
  synthetic
RKFHEKHHSHREFPFYG

SEQ ID NO: 22 - protein, 13 aa, Artificial sequence
  synthetic
EKHHSHREFPFYG

SEQ ID NO: 23 - protein, 19 aa, Artificial sequence
  synthetic
HHSHREFPFYGDYGSNYLY

SEQ ID NO: 24 - protein, 17 aa, Artificial sequence
  synthetic
SHREFPFYGDYGSNYLY

SEQ ID NO: 25 - protein, 15 aa, Artificial sequence
  synthetic
SHREFPFYGDYGSNY

SEQ ID NO: 26 - protein, 15 aa, Artificial sequence
  synthetic
FPFYGDYGSNYLYDN

SEQ ID NO: 27 - protein, 13 aa, Artificial sequence
  synthetic
FYGDYGSNYLYDN

SEQ ID NO: 28 - protein, 15 aa, Artificial sequence
  synthetic
HHSHREFPFYGDYGS

SEQ ID NO: 29 - protein, 13 aa, Artificial sequence
  synthetic
SHREFPFYGDYGS

SEQ ID NO: 30 - protein, 24 aa, Artificial sequence
  synthetic
DSHAKRHHGYKRKFHEKHHSHRGY

SEQ ID NO: 31 - protein, 14 aa, Artificial sequence
  synthetic
GYKRKFHEKHHSHR

SEQ ID NO: 32 - protein, 11 aa, Artificial sequence
  synthetic
SHREFPFYGDY

SEQ ID NO: 33 - protein, 44 aa, Artificial sequence
  synthetic
GGDSHEKRHHGYRRKFHEKHHSHREFPFYGDYGSNYLYDNLPET

(19) United States
(12) Patent Application Publication
(10) Pub. No.: US 2017/0224771 A1
(43) Pub. Date: Aug. 10, 2017

(54) HISTATINS AS THERAPEUTIC AGENTS FOR OCULAR SURFACE DISEASE

(71) Applicant: RAPID PATHOGEN SCREENING, INC., Sarasota, FL (US)

(72) Inventors: Uma Mahesh BABU, Bradenton, FL (US); Robert W. VANDINE, Montoursville, PA (US); Robert P. SAMBURSKY, Bradenton, FL (US)

(21) Appl. No.: 15/519,204

(22) PCT Filed: Oct. 8, 2015
(86) PCT No.: PCT/US15/54582

§ 371 Date: Apr. 14, 2017

Related U.S. Application Data
(60) Provisional application No. 62/064,164, filed on Oct. 15, 2014.
(60) Provisional application No. 62/064,137, filed on Oct. 15, 2014.
(60) Provisional application No. 62/064,151, filed on Oct. 15, 2014.
(60) Provisional application No. 62/065,911, filed on Oct. 20, 2014.
(60) Provisional application No. 62/065,920, filed on Oct. 20, 2014.
(60) Provisional application No. 62/065,935, filed on Oct. 20, 2014.

(51) Int. Cl. A61K 38/17; A61K 45/06; A61K 47/10

(52) U.S. Cl. CPC A61K 38/1709; A61K 47/10; A61K 45/06

(57) ABSTRACT

Histatins are used to treat ocular surface diseases such as dry eye. For example, histatins are included in eye drops, eye gels, ointment, glue, or embedded in (polymer) contact lenses. In some embodiments, peptide fragments from at least two different histatins are used. In some other embodiments, an additional therapeutic (nonhistatin) agent is used in combination with the histatins. In some embodiments, histatin 5 or fragments of histatin 5 are used in combination with an additional therapeutic (non histatin) agent. Histatins may alternatively be included as preservatives in ophthalmic preparations.

### REFERENCE TO RELATED APPLICATIONS

This application claims one or more inventions which were disclosed in Provisional Application No. 62/064,164, filed Oct. 15, 2014, entitled “FORMULATIONS FOR HISTATIN PROTECTIVES AND THERAPEUTICS”, Provisional Application No. 62/064,137, filed Oct. 15, 2014, entitled “FORMULATIONS FOR HISTATIN THERAPEUTICS”, Provisional Application No. 62/064,151, filed Oct. 15, 2014, entitled “FORMULATIONS FOR HISTATIN THERAPEUTICS”, Provisional Application No. 62/065,911, filed Oct. 20, 2014, entitled “FORMULATIONS FOR HISTATIN THERAPEUTICS” and Provisional Application 62/065,920, filed Oct. 20, 2014, entitled “FORMULATIONS FOR HISTATIN THERAPEUTICS” and Provisional Application No. 62/065,935, filed Oct. 20, 2014, entitled “FORMULATIONS FOR HISTATIN PROTECTIVES AND THERAPEUTICS”. The benefit under 35 USC §119(e) of the U.S. provisional applications is hereby claimed, and the aforementioned applications are hereby incorporated herein by reference.

### BACKGROUND OF THE INVENTION

### Field of the Invention

The invention pertains to the field of treatment of ocular surface disease such as dry eye. More particularly, the invention pertains to treating dry eye using histatins.

### Description of the Related Art

Histatins have been shown in in vitro studies to be wound healing agents from saliva. More specifically, WO 2009/087117 (and its US equivalent U.S. Patent Publication 2011/0178010), herein incorporated by reference, identified peptides of histatin, which had wound healing properties in vitro.

Histatin 1 (Hst-1) and Histatin 2 (Hst-2) have been identified as major wound-closing factors in human saliva (“Discovery of the Wound Healing Capacity of Salivary Histatins”, thesis of Menno Johannes Oudhoff, Academic Centre for Dentistry Amsterdam (ACTA), VU University Amsterdam and University of Amsterdam, The Netherlands, 2010, herein incorporated by reference). These studies were all done in vitro and cannot be translated to a finding for therapeutic or clinical use, especially since wound and disease healing are complex processes that need to be highly regulated in order to function properly.

### SUMMARY OF THE INVENTION

Histatins may be used for treatment of ocular surface diseases such as dry eye in humans and other animals. For example, histatins may be included in eye drops, eye gels, ointment, glue, embedded in (polymer) contact lenses or as a coating for punctal plugs.

In some embodiments, a composition to treat dry eye includes at least one histatin and at least one other therapeutic agent to treat dry eye. In some of these embodiments, the histatin is histatin 5 or a fragment of histatin 5. In other embodiments, the histatin includes both histatin 5 and/or a fragment of histatin 5 and histatin 1/2 (histatin 1 and/or histatin 2) and/or a fragment of histatin 1/2 (histatin 1 and/or histatin 2). In these embodiments, histatin 1/2 (histatin 1 and/or histatin 2) is preferably cyclized.

Some therapeutic agents include, but are not limited to, an IL-1 receptor antagonist, an IL-1 signaling inhibitor, a TNF-α inhibitor, a TNF-α antagonist, a TNF-α receptor antagonist, an IL-8 inhibitor, an IL-8 receptor antagonist, a kinase inhibitor, a tyrosine kinase receptor antagonist, a tyrosine kinase inhibitor, Janus kinase Inhibitor JAK-1, Janus kinase inhibitor JAK-2, Janus kinase Inhibitor JAK-3, a dual JAK/spleen tyrosine kinase (Syk) inhibitor, a calcineurin inhibitor, an androgen receptor inhibitor, an estrogen receptor inhibitor, a serotonin receptor inhibitor, a calcium activated chloride channel modulator, an anti-lymphangiogenic agent, a cycloheptathiophene ZAP-70 inhibitor, a non-steroidal anti-inflammatory drug (NSAID), a partial peptide of lacritin, an siRNA, an Integrin antagonist, an Integrin inhibitor, an IL-6 receptor antagonist, an IL-6 inhibitor, a mucin-stimulating drug, a cyclosporine emulsion, rapamycin, a neutraceutical; omega 3 oil, flax seed oil, Zeaxanthin, Lutein, Zinc, Selenium, Copper, and Squalamine.

In some embodiments, the compositions including the histatins are used to treat non-viral conjunctivitis, keratitis or infections caused by free-living bacterivores such as Acanthamoeba.

### DETAILED DESCRIPTION OF THE INVENTION

Histatins are naturally occurring oral salivary peptides produced by humans and non-human primates that demonstrate direct anti-infective activity and potent anti-inflammatory properties and stimulate epithelial wound healing in several tissue and organ culture systems. Histatins are also naturally existing protease inhibitors and may inhibit the lytic function of proteses and peptidases. A research facility has developed a technique to isolate this natural substance, making it a potential topical treatment for wounds and inflammatory diseases or conditions.

“Histatin 1/2”, as used herein, denotes histatin 1 and/or histatin 2.

U.S. Patent Publication No. 2013/0310326, published Nov. 21, 2013, entitled “HISTATIN FOR CORNEAL WOUND HEALING AND OCULAR SURFACE DISEASE” and U.S. Patent Publication No. 2013/0310327, entitled “HISTATIN FOR CORNEAL WOUND HEALING AND OCULAR SURFACE DISEASE”, both herein incorporated by reference, disclose methods of treating wounds and ocular surface disease using histatins.

Histatin 1 is known to aid in epithelial migration and wound healing. Staining in patients with dry eye indicates the existence of wounds. Histatin 5 has been shown to be an attenuator/modulator of the inflammatory cascade of cytokines such as IL-1, IL-6, IL-8, and TNF-alpha. IL-1 and IL-6 inhibitors or antagonists are currently being used to treat dry eye. Antagonists and/or inhibitors of these inflammatory markers such as these and non-specific anti-inflammatory drugs reduce the production and action of inflammatory cytokines and thus there is a reduction of deleterious cascade of inflammatory events. Including histatin 5 and/or histatin 1 in such therapeutic formulations may help alleviate the symptoms of dry eye and act synergistically with other therapeutic agents for dry eye.

In a preferred embodiment, a peptide including at least one amino acid sequence of at least eight amino acids adjacently present in Histatin 5 is used to treat an ocular surface disease such as dry eye. In some of these embodiments, the peptide is cyclized. In other preferred embodiments, multiple histatin peptides or peptide fragments are used.

In other embodiments, a composition to treat an ocular surface disease such as dry eye includes at least one histatin and at least one other therapeutic agent to treat dry eye. In some of these embodiments, the histatin is histatin 5 or a fragment of histatin 5. In some of these embodiments, histatin 5 or the fragment of histatin 5 is cyclized. In other embodiments, the histatin includes both histatin 5 and/or a fragment of histatin 5 and histatin 1/2 (histatin 1 and/or histatin 2) and/or a fragment of histatin 1/2 (histatin 1 and/or histatin 2). In these embodiments, histatin 1/2 (histatin 1 and/or histatin 2) is preferably cyclized.

In some embodiments, topically applied artificial tear products or lubricants are combined with the therapeutics (histatins and nonhistatin therapeutics) described herein. Artificial tear products typically contain hypotonic or isotonic buffered solutions containing electrolytes, surfactants and various types of viscosity agents.

In some preferred embodiments, the nonhistatin agents are anti-inflammatory drugs.

Some therapeutic (nonhistatin) agents include, but are not limited to, IL-1 receptor antagonists (for example, anakinra IL-1 receptor antagonist, sold under the trademark Kineret® by Amgen, Thousand Oaks, Calif.), IL-1 signaling inhibitors (for example, EBI-005 IL-1 signaling inhibitor, Eleven Biotherapeutics, Cambridge, Mass.), TNF-α inhibitors or antagonists or receptor antagonists, IL-8 inhibitors or receptor antagonists, kinase inhibitors, Tyrosine kinase receptor antagonists or inhibitors (for example, the tyrosine receptor antagonist MIM-D3, Mimetogen, Montreal, Canada, or the Syk-specific inhibitors PRT02761 and PRT02607, Aciex Therapeutics, Boston, Mass.), Janus kinase Inhibitors JAK 1,2,3 (for example, Tofacitinib Janus kinase inhibitor, sold under the trademark Xeljanz® by Pfizer, New York, N.Y.), dual JAK/spleen tyrosine kinase (Syk) inhibitors (for example PRT02070, a combination JAK/Syk inhibitor, Aciex Therapeutics, Boston, Mass.), calcineurin inhibitors (for example Voclosporin™calcineurin inhibitor, Lux Biosciences, Jersey City, N.J.), androgen and estrogen receptor inhibitors, serotonin receptor inhibitors, calcium activated chloride channel modulators, anti-lymphangiogenic agents, cycloheptathiophene (for example Norketotifen™ cycloheptathiophene compound, Bridge Pharma, Inc., Sarasota, Fla.), ZAP-70 inhibitors, partial peptides of lacritin, siRNAs, integrin antagonists or inhibitors (for example Lifitegrast integrin antagonist, Shire, Dublin, Ireland), IL-6 receptor antagonists or inhibitors, rapamycin, mucin-stimulating drugs (for example rebamipide, Otsuka Pharmaceuticals, Tokyo, Japan and diquafosol, Merck, Whitehouse Station, N.J.), non-steroidal anti-inflammatory drugs (NSAID), and Cyclosporin emulsions.

One example of a cyclosporine ophthalmic emulsion is Restasis® (Allergan, Inc., Irvine, Calif.). Many of these agents are specific agents, which reduce the presence of inflammatory cytokines or inhibit their deleterious actions in inflammatory conditions such as dry eye disease. Non-specific anti-inflammatory steroids, corticosteroids, loteprednol, a loteprednol etabonate ophthalmic suspension, for example the suspension marketed under the trademark Lotemax® (Bausch & Lomb Inc.), or doxycycline, cyclosporine and rapamycin could be used to treat dry eye identified before laser surgery. Some of the non-specific agents such as cyclosporin or rapamycin are toxic and their dosage may be reduced by incorporating histatin in their formulations where they can act additively or synergistically. Other therapeutic agents include neutraceuticals, which include, but are not limited to, vitamin D, Omega 3 oils, Flax seed oils, Zeaxanthin, Lutein, Zinc, Selenium, Copper, or Squalamine.

A discussion of dry eye therapeutic compounds is found in Colligris et al., “Recent developments on dry eye disease treatment compounds” Saudi Journal of Ophthalmology (2014) 28, 19-30, herein incorporated by reference, and any of the therapeutic agents discussed in Colligris et al. could be used in combination with histatins to treat ocular surface diseases such as dry eye.

Table 1 in Colligris lists some therapeutic drugs and their properties. Any of the therapeutics listed in that Table could be used in combination with a histatin. WO2011/044563 (Eleven Biotherapeutics, Inc.), entitled “FAMILY CYTOKINE COMPOSITIONS AND USES”, published Apr. 14, 2011, discloses recombinantly modified proteins that bind to an interleukin receptor IL-17R. WO2011/163452 (Eleven Biotherapeutics, Inc.), entitled “TREATING SURFACE OF THE EYE DISORDERS”, published Dec. 29, 2011, discloses administration of administration of an IL-1 or IL-17 cytokine. WO2010/047500 (Benebiosis Co. Ltd.) discloses sialic acid and independent monosaccharide residues. WO2009/064983 (Alcon Research, Ltd.), entitled “METHODS AND COMPOSITIONS FOR TREATING DRY EYE”, published May 22, 2009, discloses protease, inhibiting peptide substrates, a borate salt and a galactomannan. WO2009/114512, entitled “AZETIDINE AND CYCLOBUTANE DERIVATIVES AS JAK INHIBITORS”, published Sep. 17, 2009, WO2010/039939, entitled “a JANUS KINASE INHIBITORS FOR TREATMENT OF DRY EYE AND OTHER EYE RELATED DISEASES”, published Apr. 8, 2010, and U.S. Pat. No. 8,158,616, entitled “AZETIDINE AND CYCLOBUTANE DERIVATIVES AS JAK INHIBITORS”, issued Apr. 17, 2012 (Incyte Corporation), disclose azetidine and cyclobutane derivatives as JAK inhibitors. WO2010/085684, entitled “COMPOSITIONS AND METHODS FOR INHIBITION OF THE JAK PATHWAY”, published Jul. 29, 2010, WO2011/017178, entitled “COMPOSITIONS AND METHODS FOR INHIBITION OF THE JAK PATHWAY”, published Feb. 10, 2011, and WO2012/015972, entitled “COMPOSITIONS AND METHODS FOR INHIBITION OF THE JAK PATHWAY”, published Feb. 2, 2012, (Rigel Pharmaceuticals Inc.) disclose 2,4-Substituted pyrimidinediamine compounds as JAK inhibitors. All of the references in this paragraph are incorporated by reference herein.

WO2009/089036 (Schepens Eye Research Institute), entitled “THERAPEUTIC COMPOSITIONS FOR TREATMENT OF OCULAR INFLAMMATORY DISORDERS”, published Jul. 16, 2009, discloses physiological acceptable salt, poleaxes analogs with carpool, carpool/hydroxypropylmethycellulose (HPMC), carpool-methyl cellulose, a mucolytic agent, carboxymethylcellulose (CMC), hyaluronic acid, cyclodextrin, and petroleum. WO2010/124259, entitled “ALLOSTERAMERS FOR TNF RECEPTORS AND USES THEREOF”, published Oct. 28, 2010, and WO2010/124262 entitled “METHODS OF IDENTIFICATION OF ALLOSTERAMERS AND USES THEREOF”, published Oct. 28, 2010 (Allostera Pharma Inc.), disclose Allosteramers™ peptides, which are short peptides that can effectively modulate TNF receptor activities. WO2009/048929 (Lux Biosciences, Inc.), entitled “OPHTHALMIC COMPOSITIONS COMPRISING CALCINEURIN INHIBITORS OR MOTOR INHIBITORS”, published Apr. 16, 2009, discloses Voclosporin, a calcineurin inhibitor. U.S. Pat. Nos. 7,772,433, entitled “SARMS AND METHOD OF USE THEREOF”, issued Aug. 10, 2010, 8,080,682, entitled “SUBSTITUTED ACYLANILIDES AND METHODS OF USE THEREOF”, issued Dec. 20, 2011, and 8,110,562, entitled “SELECTIVE ANDROGEN RECEPTOR MODULATORS, ANALOGS, AND DERIVATIVES THEREOF AND USES THEREOF”, issued Feb. 7, 2012(University of Tennessee), disclose selective androgen receptor modulators (SARM) (a substituted acylanilide). U.S. Pat. No. 8,158,828 (Gtx Inc.), entitled “NUCLEAR RECEPTOR BINDING AGENTS”, issued Apr. 17, 2012, discloses selective estrogen receptor modulators (SERMs). All of the references in this paragraph are incorporated by reference herein.

WO2009/082437 (Ligand Pharmaceuticals Inc.), entitled “ELECTIVE ANDROGEN RECEPTOR MODULATORS (SARMS) AND USES THEREOF”, published Aug. 12, 2010, discloses selective androgen receptor modulators (SARMS). WO2010/092546 (Consiglio Nazionale Delle Ricerche), entitled “ANDROGEN RECEPTOR MODULATING COMPOUNDS, PREPARATION AND USES THEREOF”, published Aug. 19, 2010, discloses selective androgen receptor modulator (non-steroidal propionanilide and hydantoine structured compounds). WO2011/068786 (Bridge Pharma, Inc.), entitled “TREATING XEROPHTHALMIA WITH COMPOUNDS INCREASING MEIBOMIAN GLAND SECRETION”, published Oct. 13, 2011, discloses R-salbutamol—increasing meibomian gland secretion. U.S. Pat. No. 7,585,877 (Acadia Pharmaceuticals, Inc.), entitled “AMINOPHENYL DERIVATES AS SSELECTIVE ANDROGEN RECEPTOR MODULATORS”, issued Sep. 8, 2009, discloses selective androgen receptor modulators-aminophenyl derivatives. WO2008/153746 (Aciex Inc.), entitled “FORMULATIONS AND METHODS FOR TREATING DRY EYE”, published Dec. 18, 2008, discloses acular (ketorolac tromethamine 0.5% ophthalmic solution) with a carboxymethylcellulose (CMC)-based artificial tear. WO2009/039461, entitled “N-SUBSTITUTED PIPERIDINE DERIVATIVES AS SEROTONIN RECEPTOR AGENTS”, published Oct. 29, 2009, and WO2010/111353, entitled “N-SUBSTITUTED PIPERIDINE DERIVATIVES AS SEROTONIN RECEPTOR AGENTS”, published Sep. 30, 2010 (Acadia Pharmaceuticals, Inc.), disclose serotonin receptor agents-Serotonin or 5-hydroxytryptamine (5-HT). All of the references in this paragraph are incorporated by reference herein.

WO2009/051439 (AmorePacific Corporation), entitled “METHOD OF IDENTIFYING AGENTS WHICH MODULATE THE ACTIVITY OF CALCIUM-ACTIVATED CHLORIDE CHANNEL”, published Jul. 16, 2009, discloses a modulator of the activity of calcium activated chloride channel. WO2011/106697 (Schepens Eye Research Institute), entitled “THERAPEUTIC COMPOSITIONS FOR THE TREATMENT OF DRY EYE DISEASE”, published Sep. 1, 2011, discloses an anti-lymphangiogenic agent. WO2010/047681, entitled “TREATING XEROPHTHALMIA WITH NORKETOTIFEN”, published Apr. 29, 2010, and WO2010/059894, entitled “OCULAR FORMULATIONS OF NORKETOTIFEN”, published May 27, 2010 (Bridge Pharma, Inc.), disclose Norketotifen, a cycloheptathiophene compound. WO2010/146132 (Cellzome Limited), entitled “SULFONAMIDES AND SULFAMIDES AS ZAP-70 INHIBITORS”, published Dec. 23, 2010, discloses ZAP-70 inhibitors, pyrimidine derivatives or sulfonamides and sulfamides or heterocyclylaminopyrimidines. WO2011/034207 (Senju Pharmaceutical Co., Ltd.), entitled “PARTIAL PEPTIDE OF LACRITIN”, published Mar. 24, 2011, discloses a polypeptide having a partial sequence of lacritin. WO2012/006083 (University of Florida), entitled “TARGETED RECEPTOR-MEDIATED SIRNA”, published Apr. 12, 2012, discloses siRNAs delivery to the cell cytoplasm by endocytosis. WO2013/096226 (Sylentis S.A.), entitled “TRPV1 ANTAGONISTS”, published Jun. 27, 2013, discloses SYL1001 siRNA to target Transient Receptor Potential Vanilloid 1 (TRPV1). All of the references in this paragraph are incorporated by reference herein.

In some embodiments, at least one histatin is used to treat dry eye in patients with Sjogren's syndrome. Sjogren's syndrome is a disease in which the patient's immune system attacks the glands that makes tears and saliva, resulting in symptoms of dry mouth and dry eye. In some of these embodiments, histatin 5 or a fragment of histatin 5 is used in combination with rapamycin as a therapeutic agent to treat dry eye in Sjogren's syndrome patients. In other embodiments, histatin 5 or a fragment of histatin 5 and cyclized histatin 1 or a fragment of cyclized histatin 1 are used to treat dry eye in Sjogren's syndrome patients. In yet other embodiments, histatin 5 or a fragment of histatin 5, cyclized histatin 1 or a fragment of cyclized histatin 1, in combination with rapamycin are used to treat dry eye in Sjogren's syndrome patients.

In alternative embodiments, the compositions including the histatins and the nonhistatin therapeutic agents are used to treat non-viral conjunctivitis or keratitis or ocular infection caused by bacteriovores such as Acanthamoeba.

In one preferred embodiment, a method of treating an ocular surface disease such as dry eye includes the step of administering a therapeutic amount of at least a portion of a histatin peptide and a nonhistatin peptide to an ocular surface.

In another preferred embodiment, a method of treating an ocular surface disease such as dry eye includes the step of administering a therapeutic amount of at least a portion of at least two histatin peptides and a nonhistatin peptide to an ocular surface.

The peptides are preferably administered using eye drops, gels, ointments including histatin, tissue glue, punctal plugs, or by incorporating histatin into a contact lens worn by a patient. In some preferred embodiments, the contact lenses are biodegradable or self absorbing collagen based contact lenses. In other embodiments, the gels or ointments are biodegradable or self-absorbing collagen based gels or ointments. In other embodiments, histatins can be administered in slow releasing liposomes made of fish oil or shark oils Omega 3 or squalamine or Flax seed oils or globules of their emulsion mixtures.

Punctal plugs relieve symptoms when tear production is borderline or if the duration of the applied tear substitutes needs to be prolonged. They are helpful as adjunctive treatment in the management of dry eye disease. Therefore, histatin coated punctal plugs are very helpful in inhibiting the deleterious functions of inflammatory proteases such as MMP-9 and Cathepsin S, which would have drained out in the absence of the punctal plugs.

Dry eyes or dry eye conditions occur when tears do not sufficiently lubricate the eye. Under these conditions, patients may experience pain, light sensitivity, a gritty sensation, a feeling of a foreign body in the eye, redness, itching, and/or blurred vision. Some causes for dry eye include imbalances in the ocular tear flow system, dry air (for example low humidity environments) or other conditions that dry out the eye, wearing contact lenses, gender (generally females are more susceptible), aging (generally, adults over 40 years old are the most susceptible), side effects of drugs (for example antihistamines, antihypertensives, anticholinergics, antipsychotics, antidepressants, beta-blockers, atenolol, chlorpheniramine,hydrochlorothiazide, isotretinoin, ketorolac, ketotifen,levocabastin, levofloxacin, oxybutynin, tolterodine, or birth control pills), hormone change due to a hysterectomy or menopause, prolonged computer use (particularly when the use results in prolonged non-blinking or prolonged staring), prior surgery (such as LASIK, PRK or cataract surgery), diseases (for example secondary Sjogren's syndrome, rheumatoid arthritis, lupus, connective tissue disease, ocular inflammatory disease or collagen vascular disease, vitamin A deficit), chemical or thermal bums, trauma or injury, or eyelid problems such as blepharitis.

In some preferred embodiments, the histatin concentration is between approximately 0.1 μg/mL and approximately 10 mg/mL. In other preferred embodiments, the histatin concentration is between approximately 0.1 μg/mL and 10 μg/mL. In some preferred embodiments, the histatin concentration is greater than or equal to approximately 1 μM.

In embodiments, with histatin 5 (or fragments of histatin 5) and cyclized histatin 1 (or cyclized fragments of histatin 1), some preferred weight-to-weight ratios (Hst5:cHst1) are 1:1, 10:1, 6:1, 1:5 and 1:10 wt/wt. Preferred concentrations (combined for both histatins) range from 1 μg to 10 mg/mL. In a particularly preferred embodiment, the ratio (Hst5:cHst1) is 1:1 and the concentration is in the range of 50 μg to 100 μg for each ml.

In some embodiments, histatin 5 (or fragments of histatin 5) and cyclized histatin 1 (or cyclized fragments of histatin 1) are mixed with glycerin (0.1 to 1% and/or propylene glycol (0.1 to 1%) to form sterile eye drops. These drops may also include preservatives. In other embodiments, the histatins are in a lubricant eye gel formulation. In other embodiments, the histatins may be in a liposome type emulsion.

The administering step may be repeated multiple times per day and/or for a plurality of days. In one preferred embodiment, this step is repeated at least one time a day for a plurality of days. In another preferred embodiment, the step is repeated chronically at least one time a day. In some preferred embodiments, the step is repeated up to hourly for a plurality of days. In another preferred embodiment, the step is repeated at least two times a day for a plurality of days. In yet another preferred embodiment, the step is repeated at least three times a day for a plurality of days, for example for seven days. In another preferred embodiment, the step is repeated four times a day for five days.

In one preferred embodiment, the histatin is a peptide including 8 to 44 amino acids. In some preferred embodiments, the peptide is an L-peptide. In other preferred embodiments, the peptide is a cyclic peptide.

In some preferred embodiments, the amino acid sequence of the histatin peptide is one or more of SEQ ID NOS: 1 through 33, or any combinations of these sequences. In alternative embodiments, one or more of the amino acid sequences have a substitution, deletion and/or insertion of up to 3 amino acids. In other alternative embodiments, one or more of the amino acid sequences have a substitution, a deletion and/or an insertion of two or less amino acids. In other alternative embodiments, one or more of the amino acid sequences have a substitution, a deletion, and/or an insertion in one amino acid.

The SEQ ID NO: 4 peptide is also known as Histatin 1 (Hst-1). Note that the first serine in this amino acid sequence may be a phosphoserine. The SEQ ID NO: 5 peptide is also known as Histatin 2 (Hst-2, also equivalent to amino acids 12-38 of Hst-1). The SEQ ID NO: 6 peptide is also known as Histatin 3 (Hst-3). The SEQ ID NO: 30 peptide is also known as Histatin 5 (Hst-5). Parts and fragments of each of these amino acid sequences may be used, alone or in combination, including but not limited to SEQ ID NOS: 1-3, 7-29 (for Histatin 1, Histatin 2 and Histatin 3) and SEQ ID NO: 31 (for Histatin 5) to treat dry eye in the embodiments described herein. While the L stereoisomer of the amino acids is preferred for the amino acid sequences described herein, D stereoisomers may alternatively be used. Alternatively, amino acid sequences that include these histatins and other amino acids, for example SEQ ID NO: 33, which is a sortase cyclized histatin (including all of Histatin 1), may be used in the embodiments described herein. Any histatin sequences could be cyclized and used in the embodiments described herein.

In preferred embodiments, histatin 1 and/or histatin 2 amino acid sequences in combination with a histatin 5 amino acid sequence, and a third therapeutic (nonhistatin) agent that is known to alleviate the symptoms of dry eye has synergistic healing effects for dry eye syndrome. A “nonhistatin” therapeutic agent, as defined herein, is any ocular therapeutic peptide that has less than six contiguous amino acids of a histatin peptide sequence within it.

In other preferred embodiments, a histatin 5 amino acid sequence, in combination with a second (nonhistatin) therapeutic agent that is known to alleviate the symptoms of dry eye has synergistic healing effects for ocular surface diseases such as dry eye syndromes.

In embodiments to treat dry eye in Sjogren's syndrome patients, histatin 1 and/or histatin 2 amino acid sequences in combination with a histatin 5 amino acid sequence are used to treat dry eye. In some of these embodiments, rapamycin is also used in the treatment. Rapamycin and its derivatives have both immunosuppressant and anti-tumor properties. These rapamycin actions are mediated through the specific inhibition of the mTOR protein kinase. Therefore, a combination of histatins and rapamycin may not only be effective in autoimmune diseases such as Sjogren's Syndrome, but may alleviate any onset of dry eye disease and discomfort. In other embodiments to treat dry eye in Sjogren's syndrome patients, a histatin 5 amino acid sequence and rapamycin are used to treat dry eye.

In one preferred embodiment, a method of treating ocular surface disease such as dry eye includes the step of administering a therapeutic amount of peptide or peptide fragments of at least two histatins and a third nonhistatin therapeutic agent known to treat dry eye to an ocular surface. The histatins and the third therapeutic agent are preferably administered using eye drops, gels, ointments including histatin, tissue glue, or by incorporating histatin into a contact lens worn by a patient. In a preferred embodiment, the therapeutic amount of histatin better alleviates dry eye symptoms compared to eyes not treated with histatin. The peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

In another preferred embodiment, a method of treating dry eye includes the step of administering a therapeutic amount of peptide or peptide fragments of histatin 5 in combination with a therapeutic nonhistatin agent known to treat dry eye to an ocular surface. The histatin 5 and the therapeutic agent are preferably administered using eye drops, gels, ointments including histatin, tissue glue, or by incorporating histatin into a contact lens worn by a patient. In a preferred embodiment, the therapeutic amount of histatin better alleviates dry eye symptoms compared to eyes not treated with histatin. The amino acid sequence of histatin 5 is preferably selected from the group consisting of SEQ ID NO: 30 and SEQ ID NO: 31.

Some preferred embodiments use amino acid sequences from Hst-1 and/or Hst-2 in combination with amino acid sequences from Hst-5, and a third therapeutic nonhistatin agent, to treat dry eye. In these embodiments, one or more amino acid sequences from Hst-1 and/or Hst-2 are chosen, and one or more amino acid sequences from Hst-5 are chosen. In some embodiments, the full length Histatin 1 (SEQ ID NO: 4), full length Histatin 2 (SEQ ID NO: 5), and/or the full length Histatin 5 (SEQ ID NO: 30) could be used. In other embodiments, portions of Hst-1, Hst-2, and/or Hst-5 could be used. For example, SEQ ID NO: 29, which is equivalent to amino acids 20-32 of Histatin 1, may be a preferred amino acid sequence to use in some embodiments. In other examples, peptides including SEQ ID NO: 32, a peptide fragment of Histatin 1 and Histatin 2 that appears to be a core motif for wound closure, may be used. Other preferred sequences from Hst-1 and Hst-2 include, but are not limited to, SEQ ID NO: 8, SEQ ID NO: 9 and SEQ ID NO: 13.

As another example, SEQ ID NO: 31, a fragment of Histatin 5 (Gusman et al., “Salivary Histatin 5 is an inhibitor of Both Host and Bacterial Enzymes Implicated in Periodontal Disease”, Infect. Immun. 2001, 69 (3): 1402, pp. 1402-1408, herein incorporated by reference), may be used, preferably in combination with Histatin 1 or Histatin 2 or fragments thereof. In other preferred embodiments, fragments of Hst-1 or Hst-2 are used with full length Hst-5 (SEQ ID NO: 30), or full length Hst-1 (SEQ ID NO: 4) or Hst-2 (SEQ ID NO: 5) are used with fragments of Hst-5 (for example, SEQ ID NO: 31). In yet other embodiments, any combination of fragments of Hst-1 and/or Hst-2, full length Hst-1 and/or Hst-2, fragments of Hst-5, or full length Hst-5 may be used. In some preferred embodiments, the concentration of the Hst-5 peptide used is greater than or equal to approximately 1 μM.

Other preferred embodiments use amino acid sequences from Hst-5, in combination with another (nonhistatin) therapeutic agent, to treat dry eye. In these embodiments, one or more amino acid sequences from Hst-5 are chosen. SEQ ID NO: 30 (full length histatin 5) or SEQ ID NO: 31, a fragment of Histatin 5, may be used. In some preferred embodiments, the concentration of the Hst-5 peptide used is greater than or equal to approximately 1 μM.

Some therapeutic (nonhistatin) agents that may be used in the embodiments descried herein include, but are not limited to, an IL-1 receptor antagonist, an IL-1 signaling inhibitor, a TNF-α inhibitor, a TNF-α antagonist, a TNF-α receptor antagonist, an IL-8 inhibitor, an IL-8 receptor antagonist, a kinase inhibitor, a tyrosine kinase receptor antagonist, a tyrosine kinase inhibitor, Janus kinase Inhibitors JAK-1, 2 or 3, a dual JAK/spleen tyrosine kinase (Syk) inhibitor, a calcineurin inhibitor, an androgen receptor inhibitor, an estrogen receptor inhibitor, a serotonin receptor inhibitor, a calcium activated chloride channel modulator, an anti-lymphangiogenic agent, a cycloheptathiophene ZAP-70 inhibitor, a non-steroidal anti-inflammatory drug (NSAID), a partial peptide of lacritin, an siRNA, an Integrin antagonist, an Integrin inhibitor, an IL-6 receptor antagonist, an IL-6 inhibitor, a mucin-stimulating drug, and cyclosporine emulsions. One example of a cyclosporine ophthalmic emulsion is Restasis® (Allergan, Inc., Irvine, Calif.). Other therapeutic agents include neutraceuticals, which include, but are not limited to, vitamin D, Omega 3 oils, Flax seed oils, Zeazanthin, Lutein, Zinc, Selenium, Copper, or Squalamine.

The amino acids and the peptides described herein may include at least one functional grouping (for example, an amine and/or carboxylic group) protected with a protective grouping in some embodiments. Since the peptides are applied to eye tissue, a protected form of the peptide may be preferred to resist degradation. The form of protection needs to be biologically compatible and compatible with pharmaceutical use. Some examples include, but are not limited to, the acylation or the acetylation of the amino-terminal ends, cyclization or the amidation or the esterfication of the carboxy-terminal ends. Thus, the peptides described herein may be used in a protected form.

The peptides described herein may be made by traditional chemical synthesis, enzymatic synthesis, or any other method known in the art.

The peptides preferably include at least 8 amino acids. In one preferred embodiment, the peptides include a range of 8 to 44 amino acids, but the peptides may alternatively include more than 44 amino acids.

In one preferred embodiment, histatin 1 (Hst-1) or histatin 2 (Hst-2) in combination with histatin 5 (Hst-5), peptide fragments of Hst-1 or Hst-2 in combination with peptide fragments of Hst-5, or any combination, are used. Hst-5 inhibits production of Matrix Metalloproteases (MMPs).

The combination of the Hst-1 Hst-2 healing properties with the Hst-5 inhibiting MMPs is expected to be very effective. In some preferred embodiments, a concentration of at least approximately 1 μM of Hst-5, or a fragment of Hst-5, is used.

In one preferred embodiment, a cyclic version of SEQ ID NO: 33 is used in combination with SEQ ID NO: 30, either in a cyclized or non-cyclized form.

Histatins and nonhistatin therapeutic agents may be administered to humans or other animals with dry eye symptoms. Some methods of administration include, but are not limited to, incorporating the histatin and the nonhistatin therapeutic agent into eye drops, gels or ointments, incorporating the histatin and the nonhistatin therapeutic agent into tissue glue used to transiently seal corneal injuries, or embedding the histatin and the nonhistatin therapeutic agent into (polymer) contact lenses.

The compositions may be administered in any combination of daily treatments for any number of days in order to produce therapeutic results. In one preferred embodiment, the composition is administered at least once a day for a plurality of days. In another preferred embodiment, the composition is administered at least once a day chronically (for an extended period of time). In another preferred embodiment, the step may be repeated two, three, four, five times or more, or hourly, for a plurality of days or chronically. In one example, the administration of the composition is repeated three times a day for seven days. In another example, the composition is administered four times a day for five days.

### Testing and Treatment for Dry Eye Prior to Surgery

In some embodiments, an ocular patient is tested for dry eye disease prior to ocular surgery. In some embodiments, the ocular surgery is ocular laser surgery. In other embodiments, the ocular surgery is a non-laser ocular surgery. In some embodiments, the ocular surgery is blepharoplasty, cataract surgery, retina attachment cryoscopy, or canaloplasty for glaucoma. Although any test for dry eye disease may be used, in some embodiments, the test for dry eye is a lateral flow assay. In some embodiments, the assay detects MMP-9, an inflammatory marker that is consistently elevated in the tears of patients with dry eye disease. In some embodiments, the lateral flow assay is of the type marketed under the InflammaDry® trademark (Rapid Pathogen Screening, Inc.).

In some embodiments, one or more steps of the following procedure are followed to run a dry eye test that detects elevated levels of MMP-9 (for example, the InflammaDry® lateral flow assay): A tear sample is collected from the patient's palpebral conjunctiva by gently dabbing a sample collector in multiple locations along the palpebral conjunctiva. The eyelid is released after every 2 to 3 dabs to allow the patient to blink. This is repeated 6 to 8 times, and then the sampling fleece is allowed to rest against the conjunctiva for at least 5 seconds or until the sampling fleece is saturated with tears (5-10 mL). Adequate saturation of the sampling fleece is indicated by a pink color or glistening appearance. Next, the test is assembled by snapping the sample collector onto the provided test cassette. The assembled test is then dipped into the provided test buffer solution for 20 seconds for activation. Lastly, after 10 minutes have elapsed, the test values are read. The presence of 1 blue line and 1 red line in the test result window indicates a positive test result (matrix metalloproteinase-9 (MMP-9)≧40 ng/mL). The intensity of the red line is directly related to the amount of MMP-9 present, thus, mild dry eye is associated with fainter lines than more severe dry eye is. The presence of a red line of any intensity confirms the presence of elevated MMP-9. One blue line indicates a negative test result (MMP-9<40 ng/mL). The dry eye test is analyzed within 24 hours of activation. The dry eye test preferably has built-in procedural controls, including a blue control line. In the unlikely event that the test is not run properly or the reagents do not work, the blue control line does not appear, indicating an invalid test result.

In some embodiments, if the dry eye test result is positive, the patient is treated with an anti-inflammatory treatment prior to the laser surgery. In some embodiments, the anti-inflammatory treatment includes histatin and at least one nonhistatin therapeutic agent.

In some preferred embodiments, the anti-inflammatory treatment includes a combination of full length Histatin 1 and/or Histatin 2 with Histatin 5 in combination with at least one nonhistatin therapeutic agent. In other embodiments, the formulations include peptide fragments of one or more of Histatin 1 and/or Histatin 2, with Histatin 5 in combination with at least one nonhistatin therapeutic agent. In still other embodiments, both peptide fragments of one or more of Histatin 1, Histatin 2 or Histatin 5 and one or more of full length Histatin 1, Histatin 2 and/or Histatin 5 in combination with at least one nonhistatin therapeutic agent are used. The peptides or peptide fragments of the histatins preferably include at least two different sequences selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33. In one preferred embodiment, the anti-inflammatory includes SEQ ID NO: 30 and SEQ ID NO: 33 (cyclized).

In other preferred embodiments, the anti-inflammatory treatment includes Histatin 5 in combination with at least one nonhistatin therapeutic agent. In other embodiments, the formulations include peptide fragments of Histatin 5 in combination with at least one nonhistatin therapeutic agent. In still other embodiments, both peptide fragments of Histatin 5 and full length Histatin 5 are used. The peptides or peptide fragments of the histatin are preferably selected from the group consisting of: SEQ ID NO: 30 and SEQ ID NO: 31.

In other embodiments, the anti-inflammatory treatment includes one or more of the following anti-inflammatories: cyclosporin A, for example a 0.05% cyclosporine ophthalmic emulsion marketed under the trademark Restasis® (Allergan, Inc.), an omega-3 fatty acid, an anti-inflammatory steroid, a corticosteroid, loteprednol, a loteprednol etabonate ophthalmic suspension, for example the suspension marketed under the trademark Lotemax® (Baush & Lomb Inc.), or doxycycline. In other embodiments, the anti-inflammatory treatment may include a combination of any one or more of the above-listed anti-inflammatories with any one or more of the above-listed histatin peptides or peptide fragments.

Some other therapeutic (nonhistatin) agents include, but are not limited to, an IL-1 receptor antagonist, an IL-1 signaling inhibitor, a TNF-α inhibitor, a TNF-α antagonist, a TNF-α receptor antagonist, an IL-8 inhibitor, an IL-8 receptor antagonist, a kinase inhibitor, a tyrosine kinase receptor antagonist, a tyrosine kinase inhibitor, Janus kinase Inhibitors JAK-1, 2 or 3, a dual JAK/spleen tyrosine kinase (Syk) inhibitor, a calcineurin inhibitor, an androgen receptor inhibitor, an estrogen receptor inhibitor, a serotonin receptor inhibitor, a calcium activated chloride channel modulator, an anti-lymphangiogenic agent, a cycloheptathiophene ZAP-70 inhibitor, a non-steroidal anti-inflammatory drug (NSAID), a partial peptide of lacritin, an siRNA, an Integrin antagonist, an Integrin inhibitor, an IL-6 receptor antagonist, an IL-6 inhibitor, a mucin-stimulating drug, and cyclosporine emulsions. One example of a cyclosporine ophthalmic emulsion is Restasis® (Allergan, Inc., Irvine, Calif.). Other therapeutic agents include neutraceuticals, which include, but are not limited to, vitamin D, Omega 3 oils, Flax seed oils, Zeaxanthin, Lutein, Zinc, Selenium, Copper, or Squalamine.

In some embodiments, if the patient complains of dry eye but the test is negative, a punctal plug or a punctal occlusion is used prior to or during surgery.

### Ocular Histatin Formulations

In some embodiments, a formulation for ocular histatin includes a total of approximately 25-150 μg/mL of a combination of a histatin 1 fragment (which may be a portion of histatin 1 or full length histatin 1) and a histatin 5 fragment (which may be a portion of histatin 5 or full length histatin 5) in combination with at least one nonhistatin therapeutic agent in an applying vehicle. In some preferred embodiments, a formulation for ocular histatin in combination with a nonhistatin therapeutic agent includes a total of approximately 50-100 μg/mL of a combination of a histatin 1 fragment and a histatin 5 fragment in an applying vehicle. The fragment may include the entire histatin 1 or histatin 5 sequence, or just a portion of one or both of those sequences. The weight-to-weight ratio of histatin 1 to histatin 5 is preferably 1:1, 2:1, 3:1, 4:1, 5:1, 1:2, 1:3, 1:4, 1:5, or within a range inclusive of any two of these ratios. The amount of each histatin in the formulation is more preferably in the range of 50-75 wt % of the histatin 1 fragment (25 to 75 μg/mL) and 25-50 wt % of the histatin 5 fragment (12.5 to 50 μg/mL) with respect to the total weight of histatins in the formulation.

In some preferred embodiments of this formulation, both the histatin 1 and the histatin 5 fragment are cyclized. In other preferred embodiments, one of the fragments is cyclized. In other embodiments, neither of the fragments is cyclized. In one preferred embodiment, the histatin 1 fragment is a cyclized version of SEQ ID NO: 33. In another preferred embodiment, the histatin 5 fragment is SEQ ID NO: 30. In another preferred embodiment, the histatin 5 fragment is a cyclized version of SEQ ID NO: 30. In another preferred embodiment, the histatin 1 fragment is a cyclized version of SEQ ID NO: 33 and the histatin 5 fragment is SEQ ID NO: 30 (either in a cyclic or linear form).

In other embodiments, a formulation for ocular histatin in combination with a nonhistatin therapeutic agent includes approximately 10 μg to 10 mg/mL concentration of a histatin 5 fragment, preferably delivered in a 25 to 50 μL eyedrop. In embodiments with histatin 5 as the only histatin, the full length histatin is SEQ ID NO: 30 or the histatin 5 fragment is SEQ ID NO: 31. In another preferred embodiment, the histatin 5 fragment is a cyclized version of SEQ ID NO: 30 or SEQ ID NO: 31.

One preferred vehicle in ocular histatin formulations is 1.0% carboxymethylcellulose sodium. Other alternative or additional vehicles that may be used for stability include, but are not limited to, 0.2% hydroxypropyl methylcellulose, 0.2% glycerin, or 1% polyethylene glycol 400. In another preferred vehicle, the histatin formulations are combined with glycerin (0.1 to 1%) and/or propylene glycol (0.1 to 1%) in sterile eye drops or with preservatives.

Some preferred preservatives to be used in the formulation include, but are not limited to, sodium perborate (for example, GenAqua™ sodium perborate from Novartis, AG or Purite® sodium perborate from Allergan, Inc.), polyquatemium-1 (for example, Polyquad® preservative from Alcon Research, Ltd.), also known as polidronium chloride, or a borate/sorbitol/propylene glycol/zinc preservative (for example, SofZia® preservative from Novartis AG Corp.). Benzalkonium chloride (BAK) is preferably not used in these ocular formulations because of its potentially adverse side effects, including, but not limited to, an observed significant cytotoxicity to cultured ocular epithelial cells in vitro.

In some embodiments, the ocular histatin formulation is stable both at room temperature (25° C.) and at 45° C. for at least 90 days. One such formulation that has been tested and has been found to be stable, as determined by mass spectroscopy, for at least 90 days at 45° C. and for at least 505 days at 25° C. includes cyclized histatin 1 (SEQ ID NO: 33) at a concentration of 100 μg/mL in a commercially-available gel tears lubricating eye drop with 0.25% polyethylene glycol (PEG) in an aqueous buffered borate salt solution.

These formulations may be administered to humans or other animals with a corneal wound or ocular surface disorders. Some methods of administration include, but are not limited to, incorporating the histatin into eye drops, gels or ointments, incorporating the histatin into tissue glue used to transiently seal corneal injuries, or embedding the histatin into (polymer) contact lenses.

These formulations may be administered in any combination of daily treatments for any number of days in order to produce therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

### Histatins as Therapeutics for Dry Eye in Sjogren's Syndrome

Salivary Histatin 5 is an inhibitor of both host and bacterial enzymes implicated in periodontal disease (Gusman H., et. al., Infect Immun. 2001 March; 69 (3): 1402-8, herein incorporated by reference). According to Gusman, histatin 5 greatly inhibited the activity of host proteases such as MMP-2 and MMP-9.

The Applicants believe that histatin 5, or fragments of histatin 5, are potent inhibitors of all proteases, including not only matrix proteases but also Capthepsin A, which is implicated in Sjogren's syndrome. While the quantity or concentration of these proteases may not be reduced, the activity of them is greatly reduced by histatins.

A mouse model uses non-obese diabetic (NOD) mice, which exhibit Sjogren's syndrome symptoms such as dry eye and dry mouth. This mouse model (NOD) has also recently shown that the activity of the protease Cathepsin S is enhanced in both primary and secondary Sjogren's syndrome (Janga et al., “Longitudinal Characterizaton of Tear Fluid Cathepsin S, a Sjogren's Syndrome Biomarker, in the male Non-Obese Diabetic Mouse”, ARVO [Association for Research in Vision and Ophthalmology] Annual Meeting, May 4, 2015, Denver, Colo.; Edman et al., 2015, “Four tear biomarkers distinguish Sjogren's Syndrome patients from patients with other autoimmune diseases”, ARVO [Association for Research in Vision and Ophthalmology] Annual Meeting, May, 2015, Denver, Colo.; Hamm-Alvarez et al., “Tear Cathepsin S as a Candidate Biomarker for Sjogren's Syndrome”, Arthritis and Rheumatology, July, 2014, 66(7), pp. 1872-81, all herein incorporated by reference).

Rapamycin, a kinase inhibitor, is an immunosuppressant anti-cancer drug, and is given in food or in tablet form. Even though it is quite toxic, it is deemed a good therapy for cancer patients. Rapamycin has also been given in food to mice in “anti-aging” studies.

Histatin 5 and histatin 1 (preferably cyclized), or fragments thereof, may be used to treat dry eye in patients with Sjogren's syndrome. In some embodiments, histatin 5 (or fragments thereof), or both histatin 5 and histatin 1 (or fragments thereof) may be used in combination with rapamycin. In embodiments with rapamycin, the dosage of rapamycin is preferably lower than existing dosages, decreasing its toxicity. In some preferred embodiments, a range of approximately 0.05 to 1.0% (wt/v) rapamycin in the suspension is used. In one preferred embodiment, an approximately 0.1% (wt/v) rapamycin suspension is used. In some embodiments with histatins and rapamycin, the total concentration of histatins is in the range of approximately 0.01 to 10 mg/ml.

Some preferred ratios for cyclized histatin 1 (cHst1) in combination with histatin 5 (Hst5), with or without rapamycin, are shown in the Table below.

| cHst1:Hst5 ratio | Rapamycin | Rapamycin |
|---|---|---|
| 1:1 | − | + |
| 1:5 | − | + |
| 1:10 | − | + |
| 10:1 | − | + |
| 5:1 | − | + |

Using the NOD mouse model for Sjogren's syndrome and with the activity of Cathepsin S as the assay, the efficacy of each of these formulations may be analyzed. Since histatins are known as protease inhibitors and potently reduce the activity of MMP-2 and MMP-9, the Applicant believes that they inhibit other proteases such as Cathepsin S and other Cathepsins (example R). Cathepsin S activity is an excellent marker for Sjogren's syndrome. In some preferred embodiments, cHst1+Hst5 is in the range of 50 to 100 μg per mouse. By combining cHst1 with Hst5, cHst1 heals any epithelial damage which has already occurred prior to therapy. Assays to determine the increase in sIgA, lactoferrin or Cystatin C in Sjogren's syndrome may also be run to determine the efficacy of cHst1 and Hst5 with and without rapamycin. Rapamycin treatment alone may serve as the positive control for Cathepsin S activity reduction assay.

### Histatins as Preservatives

Since these naturally occurring polypeptides are potent protease inhibitors and by themselves do not induce any autoimmunity response or tolerogenic response, they may be ideal preservatives in ocular medicine. The pharmaceutical companies have started removing the known preservatives such as Benzalkoliums and parabens from their eye drop formulations. Instead, they have opted to make expensive and cumbersome to use single use dispensers. Histatins at 1 μg to 10 mg/mL may be incorporated in both prescription based or non-prescription (over the counter) eye drops or gels or ointments as alternative preservatives.

Histatins can act as preservatives as their protease inhibitor activity acts against the body's own proteases and not necessarily as antifungal or antimicrobial agents. In these embodiments, the histatins are anti “autoimmune” rather than anti-pathogenic intruder. Histatins can prevent autocatalysis of proteases such as MMP-9 and Cathepsin S, which convert their own Zymogen form to “active” form. In other words, histatins “preserve” the pre-cursor forms of these deleterious proteases.

In some embodiments, histatin 1 or histatin 2 (or fragments of histatin 1 or histatin 2, or any combination of fragments and full length histatin 1 or histatin 2) are a preservative in an ocular composition also including a nonhistatin ocular therapeutic agent. In other embodiments, combinations of histatin 1 or histatin 2 (or fragments of histatin 1 or histatin 2, or any combination of fragments and full length histatin 1 or histatin 2) with histatin 5 (or fragments of histatin 5) are a preservative in an ocular composition also including a nonhistatin ocular therapeutic agent.

All of the patent and nonpatent references discussed herein are incorporated herein by reference.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1. A method of treating dry eye, comprising the step of administering a therapeutic amount of at least one medicament to an ocular surface, wherein the medicament comprises:
a) a first therapeutic agent selected from the group consisting of:
at least a first histatin and a second histatin;
at least a fragment of the first histatin and a fragment of the second histatin;
at least the first histatin and at least a fragment of the second histatin; and
at least the second histatin and at least a fragment of the first histatin; and
b) a second therapeutic agent that is not a histatin or a fragment of a histatin.

2. The method of claim 1, wherein the second therapeutic agent is selected from the group consisting of:
an IL-1 receptor antagonist; an IL-1 signaling inhibitor; a TNF-α inhibitor; a TNF-α antagonist; a TNF-α receptor antagonist; an IL-8 inhibitor; an IL-8 receptor antagonist; a kinase inhibitor; a tyrosine kinase receptor antagonist; a tyrosine kinase inhibitor; Janus kinase Inhibitor JAK-1; Janus kinase inhibitor JAK-2; Janus kinase Inhibitor JAK-3; a dual JAK/spleen tyrosine kinase (Syk) inhibitor; a calcineurin inhibitor; an androgen receptor inhibitor; an estrogen receptor inhibitor; a serotonin receptor inhibitor; a calcium activated chloride channel modulator; an anti-lymphangiogenic agent; a cycloheptathiophene ZAP-70 inhibitor; a non-steroidal anti-inflammatory drug (NSAID); a partial peptide of lacritin; an siRNA; an Integrin antagonist; an Integrin inhibitor; an IL-6 receptor antagonist; an IL-6 inhibitor; a mucin-stimulating drug; a cyclosporine emulsion; rapamycin; a neutraceutical; omega 3 oil; flax seed oil; Zeaxanthin; Lutein; Zinc; Selenium; Copper; and Squalamine.

3-5. (canceled)

6. The method of claim 1, wherein the first histatin is histatin 5 and the second histatin is selected from the group consisting of histatin 1, histatin 2, a fragment of histatin 1 and a fragment of histatin 2.

7. The method of claim 6, wherein a ratio between the first histatin and the second histatin is selected from the group consisting of: 1:1, 10:1, 6:1, 1:5 and 1:10.

8. The method of claim 6, wherein a combined concentration of the first histatin and the second histatin ranges from 1 μg to 10 mg/mL.

9. The method of claim 6, wherein a ratio between the first histatin and the second histatin is 1:1 and a concentration of the first histatin and the second histatin are each 50 μg/ml to 100 μg/ml.

10. The method of claim 1, wherein the first histatin comprises an amino acid sequence selected from the group consisting of SEQ ID NO: 30 and SEQ ID NO: 31.

11. The method of claim 1, wherein the second histatin comprises an amino acid sequence selected from the group consisting of SEQ ID NO: 4; SEQ ID NO: 5; SEQ ID NO: 8; SEQ ID NO: 9; SEQ ID NO: 13; SEQ ID NO: 29; and SEQ ID NO: 33.

12. The method of claim 1, wherein the first histatin is SEQ ID NO: 30 and the second histatin is SEQ ID NO: 33.

13. The method of claim 12, wherein SEQ ID NO: 33 is cyclized.

14. A method of treating dry eye, comprising the step of administering a therapeutic amount of at least one medicament to an ocular surface, wherein the medicament comprises:
a) full length histatin 5 or a fragment of histatin 5; and
b) a therapeutic agent to treat dry eye, wherein the therapeutic agent is not a histatin or a fragment of a histatin.

15. The method of claim 14, wherein the therapeutic agent is selected from the group consisting of:
an IL-1 receptor antagonist; an IL-1 signaling inhibitor; a TNF-α inhibitor; a TNF-α antagonist; a TNF-α receptor antagonist; an IL-8 inhibitor; an IL-8 receptor antagonist; a kinase inhibitor; a tyrosine kinase receptor antagonist; a tyrosine kinase inhibitor; Janus kinase Inhibitor JAK-1; Janus kinase inhibitor JAK-2; Janus kinase Inhibitor JAK-3; a dual JAK/spleen tyrosine kinase (Syk) inhibitor; a calcineurin inhibitor; an androgen receptor inhibitor; an estrogen receptor inhibitor; a serotonin receptor inhibitor; a calcium activated chloride channel modulator; an anti-lymphangiogenic agent; a cycloheptathiophene ZAP-70 inhibitor; a non-steroidal anti-inflammatory drug (NSAID); a partial peptide of lacritin; an siRNA; an Integrin antagonist; an Integrin inhibitor; an IL-6 receptor antagonist; an IL-6 inhibitor; a mucin-stimulating drug; a cyclosporine emulsion; rapamycin; a neutraceutical; omega 3 oil; flax seed oil; Zeaxanthin; Lutein; Zinc; Selenium; Copper; and Squalamine.

16. The method of claim 14, wherein the histatin comprises an amino acid sequence selected from the group consisting of SEQ ID NO: 30 and SEQ ID NO: 31.

17. (canceled)

18. The method of claim 14, wherein the full length histatin 5 or the fragment of histatin 5 is a cyclic peptide.

19. (canceled)

20. A composition for treating dry eye comprising:
a) a first therapeutic agent comprising at least a first peptide and a second peptide selected from the group consisting of:
a first histatin and a second histatin;
a fragment of the first histatin and a fragment of the second histatin;
the first histatin and at least a fragment of the second histatin; and
the second histatin and at least a fragment of the first histatin; and
b) a second therapeutic agent that is not a histatin or a fragment of a histatin.

21. The composition of claim 20, wherein the second therapeutic agent is selected from the group consisting of:
an IL-1 receptor antagonist; an IL-1 signaling inhibitor; a TNF-α inhibitor; a TNF-α antagonist; a TNF-α receptor antagonist; an IL-8 inhibitor; an IL-8 receptor antagonist; a kinase inhibitor; a tyrosine kinase receptor antagonist; a tyrosine kinase inhibitor; Janus kinase Inhibitor JAK-1; Janus kinase inhibitor JAK-2; Janus kinase Inhibitor JAK-3; a dual JAK/spleen tyrosine kinase (Syk) inhibitor; a calcineurin inhibitor; an androgen receptor inhibitor; an estrogen receptor inhibitor; a serotonin receptor inhibitor; a calcium activated chloride channel modulator; an anti-lymphangiogenic agent; a cycloheptathiophene ZAP-70 inhibitor; a non-steroidal anti-inflammatory drug (NSAID); a partial peptide of lacritin; an siRNA; an Integrin antagonist; an Integrin inhibitor; an IL-6 receptor antagonist; an IL-6 inhibitor; a mucin-stimulating drug; a cyclosporine emulsion; rapamycin; a neutraceutical; omega 3 oil; flax seed oil; Zeaxanthin; Lutein; Zinc; Selenium; Copper; and Squalamine.

22. (canceled)

23. The composition of claim 20, wherein at least one of the first peptide and the second peptide is cyclized.

24. (canceled)

25. The composition of claim 20, wherein the first histatin is histatin 5 and the second histatin is selected from the group consisting of histatin 1, histatin 2, a fragment of histatin 1 and a fragment of histatin 2.

26. The composition of claim 20, wherein the first histatin comprises an amino acid sequence selected from the group consisting of SEQ ID NO: 30 and SEQ ID NO: 31.

27. The composition of claim 20, wherein the second histatin comprises an amino acid sequence selected from the group consisting of SEQ ID NO: 4; SEQ ID NO: 5; SEQ ID NO: 8; SEQ ID NO: 9; SEQ ID NO: 13; SEQ ID NO: 29; and SEQ ID NO: 33.

28. The composition of claim 20, wherein the first histatin is SEQ ID NO: 30 and the second histatin is SEQ ID NO: 33.

29. The composition of claim 28, wherein SEQ ID NO: 33 is cyclized.

30. The composition of claim 20, wherein a ratio between the first histatin and the second histatin in the composition is selected from the group consisting of: 1:1, 10:1, 6:1, 1:5 and 1:10.

31. The composition of claim 20, wherein a combined concentration of the first histatin and the second histatin in the composition ranges from 1μg/ml to 10 mg/mL.

32. The composition of claim 30, wherein a ratio between the first histatin and the second histatin in the composition is 1:1 and a concentration of the first histatin and the second histatin in the composition are each 50 μg/ml to 100 μg/ml.

33. The composition of claim 20, further comprising 0.1 to 1.0% glycerin.

34. The composition of claim 20, further comprising 0.1 to 1.0% propylene glycol.

35-67. (canceled)
